(19) United States
(12) Patent Application Publication
(10) Pub. No.: US 2024/0387054 A1
(43) Pub. Date: Nov. 21, 2024

(54) DATA PROCESSING APPARATUS, METHOD, AND PROGRAM

(71) Applicant: Nippon Telegraph and Telephone Corporation, Tokyo (JP)

(72) Inventors: Mariko NAKAMURA, Musashino-shi, Tokyo (JP); Atsushi FUKAYAMA, Musashino-shi, Tokyo (JP); Fumiya AKASAKA, Musashino-shi, Tokyo (JP)

(21) Appl. No.: 18/687,659

(22) PCT Filed: Sep. 6, 2021
(86) PCT No.: PCT/JP2021/032679

§ 371 Date: Feb. 28, 2024

(51) Int. Cl. G16H 50/30; G16H 10/20

(52) U.S. Cl. CPC G16H 50/30; G16H 10/20

(57) ABSTRACT

A data processing apparatus according to an embodiment includes: a storage unit that stores child-care data indicating details of child care of a caregiver; a first determination unit that performs analysis on the child-care data stored in the storage unit and determines whether or not an examination has to be conducted on whether or not the caregiver suffers from a mental disease or is at risk of disease, based on a result of the analysis; a notification unit that makes a notification for requesting answers to questions for the examination to the caregiver, when the first determination unit determines that the examination has to be conducted, an acquisition unit that acquires the answer to the question for the examination from the caregiver who had the notification from the notification unit; and a second determination unit that determines whether or not the caregiver suffers from a disease or is at risk of disease, based on the answers acquired by the acquisition unit.

### TECHNICAL FIELD

Embodiments of the present invention relate to a data processing apparatus, method, and program.

### BACKGROUND ART

A parent, particularly a mother, who is a caregiver for an infant of a child-raising household, is often exposed to a state of social isolation; however, since the mother is busy taking care of the infant every day, and thereby may not notice a change in her physical condition, she may develop postpartum depression, which is one of mental diseases related to child care.

In particular, a mother immediately after childbirth tends to suffer mental and physical disorders caused by damage to the body and disturbance of hormonal balance due to the childbirth, and thus it is necessary to pay attention to a mother's physical condition.

Postpartum depression is reported to tend to occur when social support is weak or a mother has an abused experience while the environment drastically changes in response to childbirth.

### CITATION LIST

### Non Patent Literature

Non Patent Literature 1: Postpartum depression, Journal of Japan Academy of Community Health Nursing Vol. 18 No. 2, 3, 2015 (p79-82)

### SUMMARY OF INVENTION

### Technical Problem

As a means for identifying a subject in need of support for postpartum depression, there is a method in which parents are asked to answer several questions, and risk screening for postpartum depression is performed based on the results of the answers to find out that postpartum depression has developed or there is a possibility that postpartum depression may develop.

However, parents who do not have an opportunity to be involved in the risk screening described above may overlook development or a possibility of postpartum depression.

The present invention has been made in view of the above circumstances, and objects of the present invention are to provide a data processing apparatus, method, and program capable of appropriately dealing with a disease related to child care by a caregiver.

### Solution to Problem

According to an aspect of the present invention, there is provided a data processing apparatus including: a storage unit that stores child-care data indicating details of child care of a caregiver; a first determination unit that performs analysis on the child-care data stored in the storage unit and determines whether or not an examination has to be conducted on whether or not the caregiver suffers from a mental disease or is at risk of disease, based on a result of the analysis; a notification unit that makes a notification for requesting answers to questions for the examination to the caregiver, when the first determination unit determines that the examination has to be conducted, an acquisition unit that acquires the answer to the question for the examination from the caregiver who had the notification from the notification unit; and a second determination unit that determines whether or not the caregiver suffers from a disease or is at risk of disease, based on the answers acquired by the acquisition unit.

According to another aspect of the present invention, there is provided a data processing method that is performed by a data processing apparatus, the data processing method including: a first determination unit that performs analysis on child-care data indicating details of child care of a caregiver and determines whether or not an examination has to be conducted on whether or not the caregiver suffers from a mental disease or is at risk of disease, based on a result of the analysis; a notification unit that makes a notification for requesting answers to questions for the examination to the caregiver, when the first determination unit determines that the examination has to be conducted, an acquisition unit that acquires the answer to the question for the examination from the caregiver who had the notification from the notification unit; and a second determination unit that determines whether or not the caregiver suffers from a disease or is at risk of disease, based on the answers acquired by the acquisition unit.

### Advantageous Effects of Invention

According to the present invention, it is possible to appropriately deal with a disease related to child care.

### BRIEF DESCRIPTION OF DRAWINGS

FIG. 1 is a diagram illustrating an application example of a child-care support data processing apparatus according to an embodiment of the present invention.

FIG. 2 is a sequence diagram illustrating an example of a processing operation by the child-care support data processing apparatus according to another embodiment of the present invention.

FIG. 3 is a block diagram illustrating an example of a hardware configuration of the child-care support data processing apparatus according to still another embodiment of the present invention.

### DESCRIPTION OF EMBODIMENTS

Hereinafter, an embodiment according to the invention will be described with reference to the drawings.

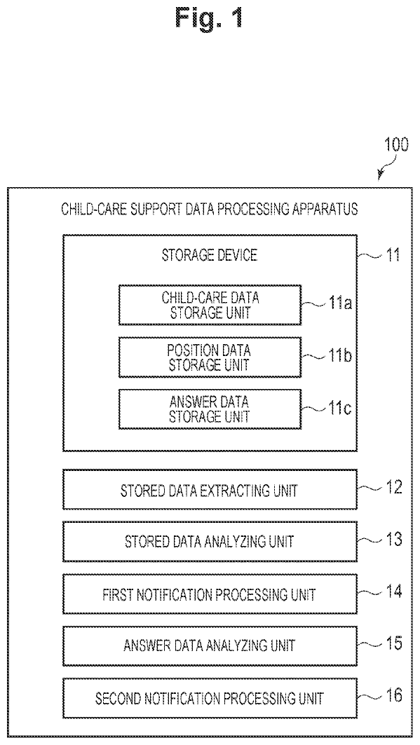

FIG. 1 illustrates an application example of a child-care support data processing apparatus according to the embodiment of the present invention.

As illustrated in FIG. 1, a child-care support data processing apparatus 100 which is a data processing apparatus according to the embodiment of the present invention includes a storage device 11, a stored data extracting unit 12, a stored data analyzing unit 13, a first notification processing unit 14, an answer data analyzing unit 15, and a second notification processing unit 16.

Functions of units of the child-care support data processing apparatus 100 may be realized by one device by being incorporated as an application program distributed or downloaded, for example, to a communications terminal such as a smartphone (hereinafter, referred to as a caregiver terminal) which can be carried by a caregiver, for example.

In addition, as another example, among the functions of the units of the child-care support data processing apparatus 100, functions such as data storage and analysis may be realized by being incorporated in, for example, a server device capable of communicating with a caregiver terminal, for example, a server device on a cloud, and all processing including interaction with the caregiver may be realized by a system in which the caregiver terminal and the server device are connected via a communication network.

The storage device 11 includes a child-care data storage unit 11a, a position data storage unit 11b, and an answer data storage unit 11c.

In the embodiment, a configuration is used in which a child-care diary application program (hereinafter, referred to as a child-care diary app) for a caregiver to record data related to a child-care diary of an infant is installed in the storage device 11, and the caregiver can register the data related to the child-care diary as electronic data depending on a screen displayed by the child-care diary app.

In addition, if a configuration is used in which the data related to the child-care diary is stored as electronic data in the storage device, instead of the configuration in which the child-care diary app is used, a website that receives registration of the data related to the child-care diary may be constructed by using the server device, and the caregiver may access the website to register the data related to the child-care diary.

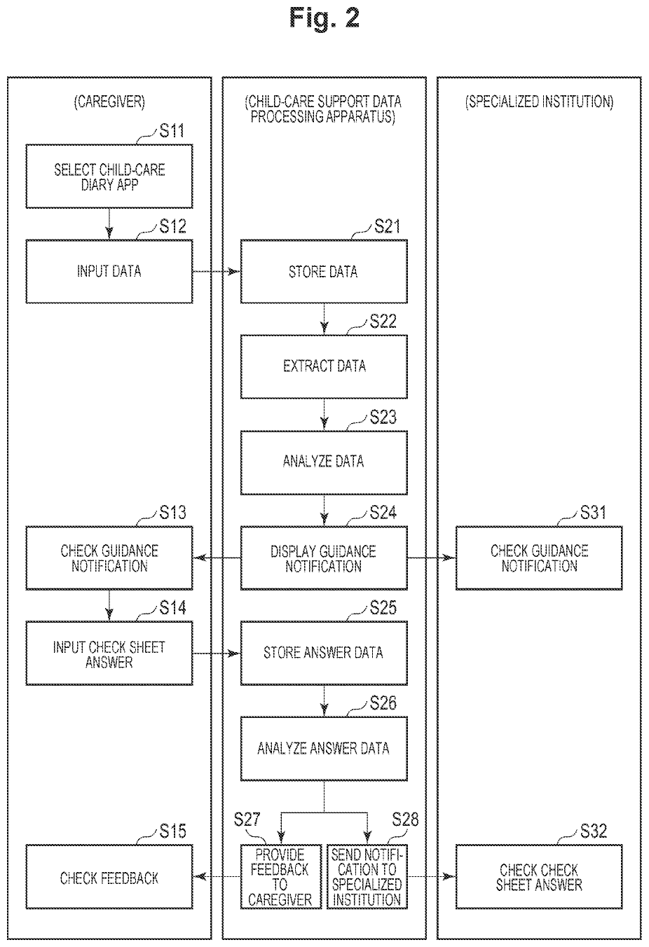

FIG. 2 is a sequence diagram illustrating an example of a processing operation by the child-care support data processing apparatus according to another embodiment of the present invention.

In the embodiment, the child-care diary app is selected and activated by an input operation to a smartphone by a caregiver (S11), and data related to the child-care diary, for example, data (hereinafter, referred to as child-care data) such as a date and time when an infant is fed, a sleep time of the infant, and a date and time and details of excretion of the infant (diaper change) or the like is sequentially input by the caregiver depending on an operation screen provided by the child-care diary app (S12), and the input data is stored in the child-care data storage unit 11a (S21).

In addition, it is assumed that a global positioning system (GPS) sensor (not illustrated) and a timer (not illustrated) are provided in the child-care support data processing apparatus 100, and on the premise that the caregiver possesses a smartphone as the child-care support data processing apparatus 100, position data sequentially detected by the GPS sensor is stored as position data of the caregiver together with time data measured by the timer in the position data storage unit 11b.

Moreover, at a predetermined timing, for example, a predetermined date and time every several days or per week, the stored data extracting unit 12 extracts data related to development of postpartum depression, the data being stored in the storage device 11 at a timing every several days or per week back from the present, for example, among the child-care data stored in the child-care data storage unit 11a and the position data stored in the position data storage unit 11b (S22).

The stored data analyzing unit 13 estimates an average, a cumulative total, or the like of sleep times of the caregiver in a predetermined period going back from the present by analyzing the child-care data and the position data extracted in S21. The sleep times can be estimated by various procedures. For example, the stored data analyzing unit 13 may estimate, as at least a part of the sleep times of the caregiver, a certain proportion of the time that is not a timing of feeding or excretion for the infant in the nighttime period and the sleep times of the infant.

In addition, the stored data analyzing unit 13 may estimate, as at least a part of the sleep time of the caregiver, for example, a certain amount of time or more in which a position indicated by the position data of the caregiver is the home and the position does not change over a certain amount of time or more in the nighttime period.

When the estimated sleep time does not satisfy criteria on the appropriate sleep time of the caregiver, the stored data analyzing unit 13 regards that a likelihood that the caregiver has insufficient sleep is relatively high and determines that an examination has to be conducted on whether or not the caregiver has developed postpartum depression or is at risk of developing postpartum depression. In addition, when the estimated sleep time satisfies the criteria, the stored data analyzing unit 13 regards that a likelihood that the caregiver has insufficient sleep is relatively low at the present time and determines that there is no need to examine whether the caregiver has developed postpartum depression or is at risk of developing postpartum depression at the present time (S23).

When the stored data analyzing unit 13 determines that the above-described examination has to be conducted, the first notification processing unit 14 transmits a message for guiding the caregiver to fill in a check sheet related to risk screening to the caregiver terminal, in order to examine the caregiver by the risk screening related to postpartum depression, and causes the message to be displayed on a display screen (not illustrated) of the caregiver terminal (S24).

In addition, the first notification processing unit 14 can transmit, to a communication terminal (hereinafter, referred to as a specialized institution terminal) installed in a specialized institution such as a midwife, an obstetrics department, or a municipality, which can care for a caregiver who develops or is at risk of developing postpartum depression, a message indicating that it is determined that the caregiver needs to examine whether or not the caregiver has developed postpartum depression or is at risk of developing postpartum depression, and a message indicating that a message (hereinafter, referred to as guidance notification) for guiding the caregiver to fill in the check sheet related to risk screening for postpartum depression is transmitted to the caregiver, to the specialized institution terminal, separately from the display to the caregiver.

The timing at which this message is transmitted may be, for example, a timing at which it is determined at a regular timing such as once a week whether or not the average or the cumulative total of the sleep times of the caregiver in the predetermined period satisfies the criteria on the appropriate sleep time, and this determination is made, or may be a timing at which it is sequentially determined whether or not the average or the cumulative total of the sleep times of the caregiver in the predetermined period satisfies the criteria on the appropriate sleep time, and this determination is made.

Consequently, the message can be displayed on a display screen (not illustrated) in the terminal to be checked by a person in charge of the specialized institution (S31).

Examples of a scale related to risk screening for postpartum depression include Edinburgh Postnatal Depression Scale (EPDS) and Hospital Anxiety Depression Scale (HADS; HAD scale), Stait-Trait Anxiety Inventory (STAI), and Whooley Questions (two question items).

In the embodiment, it is assumed that a check sheet related to risk screening is filled in online. In filling in the check sheet, a scale to be applied among the scales may be determined by the first notification processing unit 14 on the basis of a condition such as a period from postpartum or may be selected from a plurality of scales that satisfy a condition such as the period from postpartum through an input operation performed by a caregiver.

The first notification processing unit 14 causes the display screen of the caregiver terminal to display an input screen to the check sheet according to the determined or selected scale. Consequently, the caregiver can check the guidance notification related to the check sheet (S13).

In accordance with this screen, when the caregiver inputs answers to questions item on the check sheet through an input operation (S14) and performs an operation related to an end of the input, the details of the input answer is stored in the answer data storage unit 11c of the storage device 11 as answer data together with data such as an answer date and time (S25).

Moreover, the answer data analyzing unit 15 analyzes the answer data accumulated in the answer data storage unit 11c to determine whether or not a likelihood that the caregiver has developed postpartum depression or is at risk of developing postpartum depression is relatively high (S26). The criteria for this determination are based on a type of scale of the risk screening described above.

Moreover, the second notification processing unit 16 transmits, to the caregiver terminal, a message for feedback of the analysis result in S26 to the caregiver and displays the message on a display screen (not illustrated) in the caregiver terminal (S27). A timing at which the message for the feedback is transmitted may be a timing at which it is determined at a regular timing such as once a week whether or not the likelihood that the caregiver develops or is at risk of developing postpartum depression is relatively high as described above and this determination is made, or may be a timing at which it is determined sequentially whether or not the likelihood that the caregiver develops or is at risk of developing postpartum depression is relatively high as described above, and this determination is made.

Consequently, the caregiver can check the feedback, that is, whether or not the likelihood that the caregiver develops or is at risk of postpartum depression, of the answer related to the check sheet (S15).

In addition, the second notification processing unit 16 transmits a message notifying the specialized institution of the answer data accumulated in the answer data storage unit 11c to the specialized institution terminal, separately from the feedback to the caregiver, and displays the message on the display screen of the terminal (S28), so that a person in charge of the specialized institution can check the message (S32).

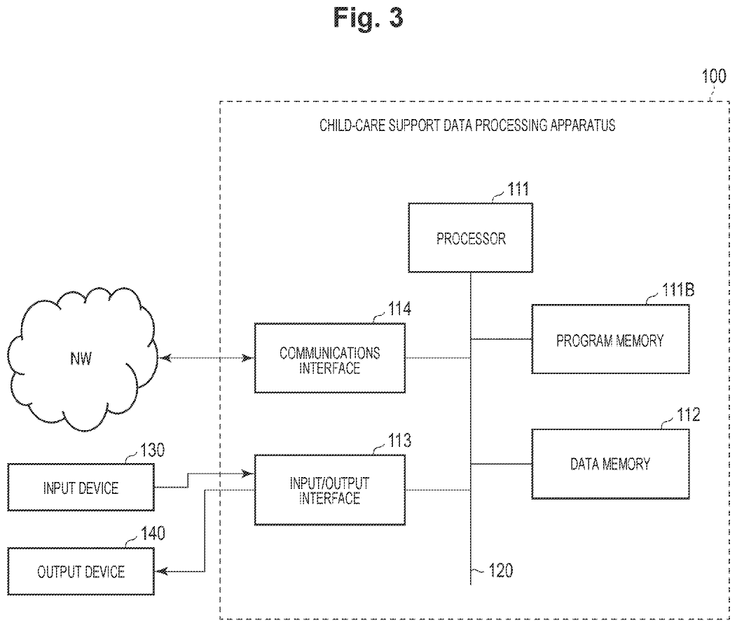

FIG. 3 is a block diagram illustrating an example of a hardware configuration of the child-care support data processing apparatus according to still another embodiment of the present invention. In the example illustrated in the drawing, the child-care support data processing apparatus 100 is configured of, for example, a server computer, a personal computer, or a portable information terminal, and includes a hardware processor 111 such as a central processing unit (CPU). Moreover, a program memory 111B, a data memory 112, an input/output interface 113, and a communications interface 114 are connected to the hardware processor 111 via a bus 120.

The communications interface 114 includes, for example, one or more wireless communications interface units and enables transmission and reception of information to and from a communication network NW. As a wireless interface, for example, an interface employing a low-power wireless data communication standard such as a wireless local area network (LAN) can be used.

An input device 130 and an output device 140 for an operator attached to the child-care support data processing apparatus 100 are connected to the input/output interface 113. The input/output interface 113 performs a process of fetching operation data input by the operator through the input device 130 such as a keyboard, a touch panel, a touchpad, or a mouse, and outputting output data to the output device 140 including a display device manufactured using liquid crystal, organic electro-luminescence (EL), or the like to display the output data. Further, as the input device 130 and the output device 140, a device installed in the child-care support data processing apparatus 100 may be used, or an input device and an output device of another information terminal that can communicate with the child-care support data processing apparatus 100 via the communication network NW may be used.

The program memory 111B is used as a non-transitory physical storage medium in a combination of a non-volatile memory such as a hard disk drive (HDD) or a solid state drive (SSD) which is writable and readable at any time and a non-volatile memory such as a read only memory (ROM), for example, and thus the program memory can store programs necessary for executing various processes according to an embodiment.

The data memory 112 is used as a physical storage medium in a combination of the above-described non-volatile memory and a volatile memory such as a random access memory (RAM), for example, and thus the data memory can be used to store various items of data acquired and created in a process of performing various kinds of processing.

The child-care support data processing apparatus 100 according to an embodiment of the present invention can be configured as a data processing apparatus including the storage device 11, the stored data extracting unit 12, the stored data analyzing unit 13, the first notification processing unit 14, the answer data analyzing unit 15, and the second notification processing unit 16 illustrated in FIG. 1 as processing function units by software.

In addition, a working memory used for various processes in the child-care support data processing apparatus 100 and various storage units in the storage device 11 illustrated in FIG. 1 can be configured using the data memory 112 illustrated in FIG. 3.

All of the processing function units in the stored data extracting unit 12, the stored data analyzing unit 13, the first notification processing unit 14, the answer data analyzing unit 15, and the second notification processing unit 16 illustrated in FIG. 1 can be realized by causing the hardware processor 111 to read and execute the program stored in the program memory 111B. Further, some or all of these processing functional units may be realized by other various modes including an integrated circuit such as an application specific integrated circuit (ASIC) or a field-programmable gate array (FPGA).

In the embodiment described above, the child-care data indicating the details of child care by the caregiver is analyzed, whether or not the examination has to be conducted on whether or not the caregiver suffers from a mental disease or is at risk of disease is determined on the basis of a result of the analysis, and when it is determined that the examination has to be conducted, a notification for requesting answers to questions for the examination is given to the caregiver, and whether or not the caregiver suffers from a disease or is at risk of disease is determined on the basis of the answer to the question for the examination by the caregiver.

Consequently, it is accurately determined that a mother who is a caregiver develops or is at relatively high risk of developing postpartum depression, and the caregiver and the specialized institution are notified. Therefore, early detection and support of postpartum depression can be expected, and early recovery or prevention of postpartum depression can be realized. Therefore, it is possible to take an appropriate action against a disease related to child care by a caregiver.

In the embodiment, an example has been described for the prevention of postpartum depression related to infant care by a mother, but the present invention is not limited thereto. For example, as long as it is a mental disease related to child care by a caregiver, the present invention can be applied to infant-care neurosis in early childhood after infancy. In this case, instead of the answer to the check sheet of the risk screening related to postpartum depression, the caregiver may answer to a check sheet for the infant-care neurosis or the like, and feedback may be transmitted to the caregiver on the basis of the answer.

In addition, the methods described in the embodiments can be stored as a program (software means) that can be executed by a computing machine (computer) in a recording medium such as a magnetic disk (floppy (registered trademark) disk, hard disk, etc.), an optical disc (CD-ROM, DVD, MO, etc.), or a semiconductor memory (ROM, RAM, flash memory, etc.) and can be transmitted and distributed by a communication medium. Further, a program stored on a medium side also includes a setting program for configuring, in the computing machine, software means (including not only an execution program but also a table and a data structure) to be executed by the computing machine. The computing machine that implements the apparatus executes the above-described processing by reading the program recorded in the recording medium, constructing the software means by the setting program as needed, and controlling an operation by the software means. Further, the recording medium described in this specification is not limited to a recording medium for distribution but includes a storage medium such as a magnetic disk or a semiconductor memory provided in a computing machine or in a device connected via a network.

The present invention is not limited to the above-described embodiments, and various types of modifications can be made at an implementation stage without departing from the gist of the invention. In addition, the embodiments may be implemented in a combination as required, and in that case, effects can be obtained by combination. Furthermore, the above-described embodiments include various types of inventions, and various types of inventions can be extracted by a combination selected from a plurality of disclosed components. For example, even if some components are eliminated from all the components described in an embodiment, a configuration from which the components are eliminated can be extracted as an invention in a case where the problem can be solved and the advantageous effects can be obtained.

### REFERENCE SIGNS LIST

100 Child-care support data processing apparatus

11 Storage device

11a Child-care data storage unit

11b Position data storage unit

11c Answer Data storage unit

12 Stored data extracting unit

13 Stored Data analyzing unit

14 First notification processing unit

15 Answer Data analyzing unit

16 Second notification processing unit

1. A data processing apparatus comprising:
a storage unit that is configured to store child-care data indicating details of child care of a caregiver;
a first determination unit, including one or more processors, configured to perform analysis on the child-care data stored in the storage unit and determine whether or not an examination has to be conducted on whether or not the caregiver suffers from a mental disease or is at risk of disease, based on a result of the analysis;
a notification unit, including one or more processors, configured to generate a notification for requesting answers to questions for the examination to the caregiver, in response to determining that the examination has to be conducted,
an acquisition unit, including one or more processors, configured to acquire the answer to the question for the examination from the caregiver who had the notification from the notification unit; and
a second determination unit, including one or more processors, configured to determine whether or not the caregiver suffers from a disease or is at risk of disease, based on the acquired answer.

2. The data processing apparatus according to claim 1, wherein the first determination unit is configured to determine a sleeping state of the caregiver based on the result of the analysis and determine whether or not the examination has to be conducted, based on the determined sleeping state.

3. The data processing apparatus according to claim 1,
wherein the child-care data includes at least one type of data among data indicating timings of feeding an infant by the caregiver, sleeping of the infant, and excretion of the infant, and
wherein the first determination unit is configured to perform analysis on the data indicating the timings, determine a sleep time of the caregiver based on a result of the analysis, and determine whether or not the examination has to be conducted, based on the determined sleep time.

4. The data processing apparatus according to claim 1,
wherein the storage unit further configured to store position data of the caregiver, and
wherein the first determination unit is configured to perform analysis on the child-care data and the position data and determine whether or not the examination has to be conducted, based on a result of the analysis.

5. The data processing apparatus according to claim 1,
wherein the notification unit is configured to notify at least one of the caregiver and a person who can provide support regarding the disease of the caregiver, in response to determining that the caregiver suffers from a disease or is at risk of disease.

6. A data processing method that is performed by a data processing apparatus, the data processing method comprising:
performing analysis on child-care data indicating details of child care of a caregiver;
determining whether or not an examination has to be conducted on whether or not the caregiver suffers from a mental disease or is at risk of disease, based on a result of the analysis;
generating a notification for requesting answers to questions for the examination to the caregiver, in response to determining that the examination has to be conducted;
acquiring the answer to the question for the examination from the caregiver who had the notification; and
determining whether or not the caregiver suffers from a disease or is at risk of disease, based on the acquired answer.

7. The data processing method according to claim 6, further comprising:
determining a sleeping state of the caregiver based on a result of the analysis; and
determining whether or not the examination has to be conducted, based on the determined sleeping state.

8. A data processing program for causing a processor to function as each of the units of the data processing apparatus according to claim 1.
